(19) United States
(12) Patent Application Publication
(10) Pub. No.: US 2011/0251520 A1
(43) Pub. Date: Oct. 13, 2011

(54) Fall-risk Evaluation and Balance Stability Enhancement System and method

(75) Inventors: Jiann-Shing Shieh, Taipei City (TW); Bernard C. Jiang, Taoyuan County (TW); Kai-Hung Wang, Taipei City (TW); Wen-Hung Yang, Taoyuan County (TW)

(73) Assignee: YUAN ZE UNIVERSITY, Taoyuan County (TW)

(21) Appl. No.: 12/757,010

(22) Filed: Apr. 8, 2010

(51) Int. Cl. A61B 5/103

(52) U.S. Cl. 600/587

(57) ABSTRACT

A Fall-risk Evaluation and Balance Stability Enhancement System and method evaluate and treat the sense of body balance. It is focused on measuring the center of pressure (COP) information, evaluating fall risk by the noninvasive physiological signals machines, and improving the body's balance and stability by stimulating physical treatment in feet. The vibratory shoes could produce physical stimulation and generate the positive treatment on the patients who have poor sense of balance to achieve the goal of reducing the risk of falls

### BACKGROUND OF THE INVENTION

1. Field of the Invention

This invention relates generally to an apparatus and a method adapted to fall-risk evaluation and balance stability enhancement. It was used a non-invasive measurement of physiological signals machine to measure physiological messages and give physical therapy via vibrating insoles that improve the body's balance system.

2. Descriptions of the Prior Art

Falls are a major public health concern [1] among the old-aged people. The growing populations of adults, like 65 years or older, increase the costs in medical care. Unintentional injuries happened at home cost U.S. society at least $217 billion in 1998 [2]. The total medical cost of fall is $90.5 billion, and about 42% of the unintentional injuries happened at home. According to the survey by the statistics department in the ministry of the interior Taiwan, the population of 65 year-old adults or older is 1.27 million in 1990 and 2.26 million in 2006 [3]. Based on the survey conducted by department of Health, Executive Yuan, R.O.C (Taiwan) [4], there were 2383 elderly died of injuries due to accidents. Thus, prevention of falls happened on elderly is an important issue for our concern.

Older adults demonstrate increased risk of fall because of physiological changes associated with balance impairment [5]. There are many characteristics causing elderly a presumed fall [6]. These characteristics are listed as follows:

| Static characteristics | Dynamic characteristic |
|---|---|
| History | Assess for acute changes in |
| Dementia | Vital signs |
| Parkinson's disease | Level of consciousness |
| Subdural hematoma | Neurological system |
| Head trauma/traumatic brain injure | Gait or balance instability |
| Hip fracture | Skin integrity |
| Osteoporosis | Skin tear |
| Gait or balance impairment | Hematoma |
| Visual impairment | Bruises |
| Hearing loss | Musculoskeletal system |
| Orthostatic hypotension | Sprain |
| Delirium | Strain |
| Medications | Fracture |
| Vasodilators | |
| Neurouleptics | |
| Agents that lower blood pressure | |
| Narcotic analgesia | |
| Diuretics | |
| Behavior associated with dementia | |
| Wandering and elopement | |
| Agitation and restlessness | |
| Visual hallucination | |
| Motor or gait apraxia | |

The static characteristics composed with medical history, medication, and behaviors associated with dementia are unchangeable and unavoidable. Any older adult with one or more conditions of static characteristics should be more careful on the prevention. The dynamic characteristics may change everyday and cause an unpredictable fall. In order to reduce the harm from these characteristics in elderly, the health care system suggested developing a system which can evaluate and treat the sense of body balance.

The reverent studies are discussed as follows. First, fall is a common accident happened among the elderly, and it often causes injuries. It most frequently occurs during walking and is associated with the chronic deterioration in neuromuscular and sensory systems, as well as with ankle muscular weakness and lower endurance of these muscles to fatigue [7]. Fall is a major cause of morbidity and mortality in the U.S. Several factors can contribute to injuries resulting from tripping, slipping and falling during locomotion such as the surface condition, transitions and the degree of walkway evenness [8]. Recently, biomechanical analyses have been applied to develop treatment programs for many lower extremities orthopedic and neurological injuries [9].

The center of pressure (COP) is the projection on the ground plane of the center of the vertical force distribution. In effect, the COP is the location where the resultant force vector would act if it could be considered to have a single point of application [10]. The COP position can be obtained directly from force plate data during posture or gait trials [11]. The function of the center of pressure (COP) is discriminating between different impairment etiologies to assess lower extremity prosthesis alignment, and moreover, for assessing gait changes via orthoses intervention. An important strength of COP calculation is that it is a single measurement which incorporates information from several forces and moments. This may be clinical significance when assessing the sources of the fall [9].

Second, postural stability is defined as the ability to maintain or control body center of mass (COM), and it is in relation to the base for preventing falls and completing desired movements. The center of pressure (COP) is an indirect measurement of postural sway and it is measured in anterior-posterior (A-P) and medial-lateral (M-L) planes [12]. An efficient nonlinear analysis method characterizes postural steadiness. The analyzed signal is the displacement of the centre of pressure (COP) collected from a force plate used for measuring postural sway [13]. Postural performance is often characterized by using the center of pressure (COP), but force distribution under the feet may provide additional information on the state of the postural control system. Foot position and orientation can also be extracted from force distribution without the need for manually tracing footprints. A low-cost force distribution measurement system is being developed to complement an existing dual force-plate platform [14].

Consequently, previous studies have developed methods to estimate the center of gravity position from the center of pressure data. The results of three pressure-based centers of gravity estimations methods are compared with kinematically determined center of gravity positions in humans. The center of pressure position varies about the center of gravity position and has higher frequency content than the motion of the center of gravity.

The performance of the adaptive autoregressive model was in comparison to that of the Fast Fourier Transform (FFT). Peak frequency was observed to provide a better resolution than mean frequency [12] and the nonlinear dynamics of the intrinsic mode functions (IMF) of the COP signal. The nonlinear properties are assessed through the reconstructed phase spaces of the different IMFs [13].

Sensory feedback is necessary for postural adjustments and facilitates to control of compensatory stepping reactions evoked by postural perturbation [15-17]. Cutaneous joint and muscular mechanoreceptors provide the necessary proprioceptive inputs [18]. Mechanical stimulation of foot mechanoreceptors can be used to perturb the proprioceptive feedback and to assess its role in generation of parkinsonian gait. The foot pressure activates the plantar mechanoreceptors that mediate postural adjustment during the stance phases of the step [17]. Several studies explored the effects of mechanical stimulation upon static balance as a mean for proprioceptive feedback modulation. This effect was attributed to enhance proprioceptive feedback. The effect of the suprathreshold stimulation is complex and depends on the frequency, amplitude, and location of the stimulation [19, 20]. Vibratory stimulation of rear foot zones has a similar effect but with an opposite direction of the body tilt. Simultaneous activation of both forefoot and rear foot zones has no effect on body tilt but does cause the center of pressure (COP) oscillations. These results imply that characteristic postural responses may be specific to the localization and stimulus characteristics.

Related filed patents tried to solve the issue of postural responses are listed as follows:
1. U.S. Pat. No. 4,814,661, Systems for measurement and analysis of forces exerted during human locomotion.

This system includes piezoelectric sensor elements advantageously mounted between supporting hard plastic material of various types or other mechanically similar materials. The sensor can further be supported by backup plate structures. Some backup plates show full shoe coverage and others show partial sole coverage. Detachable mounting formed by mechanically integrated parts allows transducer inserts to be made in a modular manner for using in different sensor shoes or sensor insole pads, thus allowing many more patients to be analyzed and treated through the use of the invention. A digital charge signal integrator circuit is also disclosed in this patent.
2. U.S. Pat. No. 5,836,899, Vibrating massage system for footwear

This massage system incorporates a battery operated power supply, which is mounted in a compartment formed in the tongue of a shoe. The power supply is connected to vibrators, which are mounted in circular beds formed in the sole of the shoe. The power supply and the vibrators are connected by wires, which are concealed in a flap formed in the upper portion of the shoe. The wires are also disposed in air canals, which are formed in the sole of the shoe. The location of the power supply in the tongue of the shoe maintains the normal balance of the shoe and enables a user to benefit from vibrating massage while standing, sitting, walking or running
3. U.S. Pat. No. 6,237,256, Balance-enhanced insert for footwear

This article of footwear and insert are provided with a means for stimulating cutaneous pressure sensation from the perimeter of the plantar surface (sole) of the foot. Increasing cutaneous sensation from the perimeter of the plantar foot surface provides the central nervous system of the wearer of the footwear with an increased ability to detect and react to shifts of the body's center of gravity toward the edges of the feet which, if left uncorrected, could result in a loss of balance or a fall. An insole is disclosed, which is provided with an elongate member or a series of protrusions, which form a narrow ridge in close proximity to the perimeter of the insole and the ridge is adapted to protrude upwardly against the plantar foot surface along at least a portion of the perimeter.
4. U.S. Pat. No. 6,273,863, Adaptive weight bearing monitoring system for rehabilitation of injuries to the lower extremities

This invention is a portable, self-learning adaptive weight bearing monitoring system for personal use during rehabilitation of orthopedic patients with fractures of the lower extremities. The system includes a flexible insole which is worn inside the shoe. The insole includes pressure and/or force sensor that measures the ground reaction force (GRF) applied at key bearing points under the foot or other portions of the patient's lower extremity. The sensors are, in turn, connected through an A/D converter to a CPU that is connected to drive a stimulator that delivers closed-loop sensory stimulation (electrical, mechanical, and/or audio) as feedback to encourage the patient to load the optimal target weight for the limb for which the weight bearing force is being measured. Accurate real-time monitoring of the weight bearing during physical rehabilitation is also provided, and, through the use of a closed-loop sensory stimulation, the patient is given continuous feedback for improving rehabilitation.
5. U.S. Pat. No. 6,507,757, Apparatus for electrical stimulation of the body

This stimulator for stimulating the leg or other parts of the body e.g. the leg in a patient with drop foot is provided. The stimulator is controlled by a foot switch and the foot switch is controlled by a user. The foot switch has to work in adverse environmental conditions and is subject to repeated use so that its characteristics vary with time. The invention provides a functional electrical stimulator for attachment to the leg that has adaptive characteristics. This invention comprises first and second electrodes for attachment to the leg to apply an electrical stimulus, a foot switch for sensing foot rise or foot strike, a circuit responsive to said foot switch for generating stimulation pulses, and means forming part of the said circuit for responding to changes in the resistance characteristics of the said switch means by adjusting a corresponding response threshold of the said circuit. The invention also provides a two-channel stimulator that offers various possibilities for controlling the signals to be supplied to different muscle groups. For example, means may be provided defining a signal pathway between the first and second channels so that the supply of stimulation pulses in one of the said first and second channels can be controlled by the state of switch means associated with the other of said first and second channels. In a further embodiment means defining a signal pathway between the first and second channels is arranged so that the supply of stimulation pulses in one of the said first and second channels can be controlled by the state of activity of the other of said first and second channels. In a yet further embodiment, the first channel is arranged to cause the stimulation pulses to time-out after a predetermined period and the second channel having no or disabled timing means so that supply of stimulation pulses is continuous in a predetermined state of limb position responsive switch means, which is associated with that channel. The two-channel stimulator can be used to treat bilateral dropped foot condition.
6. U.S. Pat. No. 7,163,517, Electro-stimulation feet acupuncture channel physiotherapy shoe

This electro-stimulation feet acupuncture channel physiotherapy shoe comprised a pair of physiotherapy shoes and an accompanying physical treatment appliance. Conductive metal wires are directly embedded along the interior extent of the shoe body. A small hole is formed in the rear surface of each shoe body. A surrounding edge of the shoe body is disposed along the periphery of the sole, and three-dimensional acupressure point protrusions and massage protrusions with built-in wire wound coils that provide for electrical conductance are relieved along the instep. The physical treatment enables the physiotherapy shoe to become positively and negatively conductive, causing the generation of low and medium frequency electric waves that have physical therapy and health care effectiveness at the reflex areas along the soles of the feet.
7. U.S. Pat. No. 7,243,446, Method for providing an insole for footwear for increased sensory stimulation and an insole suited for the method

This method provides an insole to increase sensory stimulation of a foot in the footwear. The method comprises preselecting positions on the foot with nerves at these positions to be stimulated and providing a means for stimulating elevation of the said insole at said preselected positions during step movement of said foot on the said insole.
8. U.S. Pat. No. 7,310,564, Arrangement and method for producing therapeutic insoles

This patent relates to an arrangement for producing therapeutic insoles, consisting of a platform with a scanner for scanning the undersides of the patient's feet, a data processing connection for transmitting the scanned image to a computer, a data processing computer program which converts the transmitted data into working instructions for the control of a milling machine, and of said milling machine which mills a therapeutic insole from a blank according to said working instructions, whereby neurological preceptors are placed on the scanner under the patient's foot in accordance with the patient's predisposition, whereby the preceptors are part of a set of standardized preceptors having various dimensions, whereby the preceptors are identified by markings on their underside, and whereby the scanned image contains the identifying markings and the orientation of the preceptors.

### SUMMARY OF THE INVENTION

In order to reduce the harm from these characteristics in elderly, the health care system suggested to develop a system which can evaluate and treat the sense of body balance. Therefore, this patent is focused on measuring the center of pressure (COP) information and to evaluate fall risk by the noninvasive physiological signal machines and improve the body's balance and stability system by stimulating physical treatment in feet. The vibratory shoes could produce physical stimulation and generate the positive treatment on the patients who have a poor sense of balance to achieve the goal of reducing the risk of falls.

This invention is focused on the usage of the pressure-measured instruments to collect statistics from a number of subjects of different ages. Also, we use some mathematical analysis methods, such as Multi-scale Entropy (MSE), with empirical mode decomposition (EMD) to estimate their balance and stability systems. The subjects made progress due to physical stimulation treatment by wearing vibrating insoles.

We expect the results of this invention to advance the accuracy of this subject, and the elderly will be able to obtain better medical care and not be among the high-risk groups of falling.

### BRIEF DESCRIPTION OF THE DRAWINGS

Please refer to the following figures for technical details of this invention. The figures include as follows:

FIG. 1 Vibratory Shoes;

FIG. 2 the front of the vibratory insole (1) the back of the vibratory insole;

FIG. 3 Schematic diagram of structure of vibratory shoes;

FIG. 4 The process of vibratory devices turn-on;

FIG. 5 Block diagram of the experiment procedure;

FIG. 6 Illustration of the sifting processes: (a) the original data; (b) the data in thin solid line, with the upper and lower envelopes in dot-dashed lines and the mean in thick solid line; (c) the difference between the data and m₁. This is still not an IMF, for there are negative local maxima and positive minima suggesting riding waves;

FIG. 7 The resulting empirical mode decomposition components from the typical time series data;

FIG. 8 The COP data (X-axis, Y-axis);

FIG. 9 Use EMD for detrend the COP signals;

FIG. 10 The multi-scale entropy (MSE) values: Red line is MSE value in X-axis (horizontal), Green line is MSE value in Y-axis (vertical);

FIG. 11 MSE analysis of the center of pressure from five groups with different age, Analysis of the data for the horizontal (X-axis). Subjects divided into children (under 20-year-old age), youth (20˜40 years), adults (40˜50 years), middle-aged (50˜60 years old) and elderly (over sixty-year-old above);

FIG. 12 MSE analysis of the center of pressure from five groups with different age, Analysis of the data for the vertical (Y-axis). Subjects divided into children (under 20-year-old age), youth (20˜40 years), adults (40˜50 years), middle-aged (50˜60 years old) and elderly (over 60 year-old above);

FIG. 13 The one of the subject's MSE data from before and after wearing the vibratory shoes (Red line is before wearing vibratory shoes and Green line is after wearing vibratory shoes.);

FIG. 14 MSE area curve in X-axis in five noises levels from one subject;

FIG. 15 MSE area curve in Y-axis in five noises levels from one subject.

### DETAILED DESCRIPTION OF THE PREFERRED EMBODIMENT

### System Description

The Fall-risk Evaluation and Balance Stability Enhancement System and method divided into two parts: treatment and assessment system.

(1) Treatment System

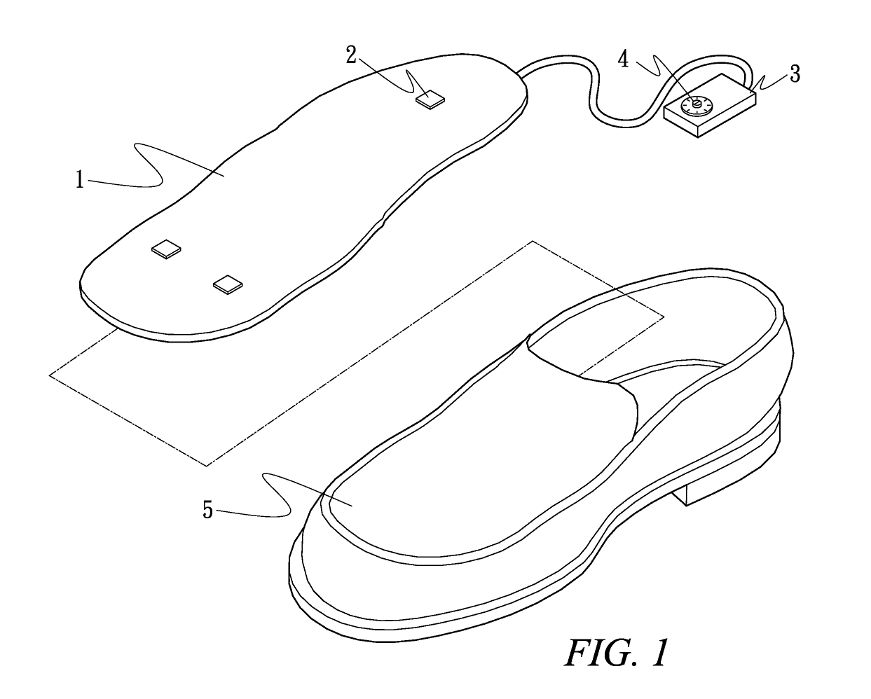

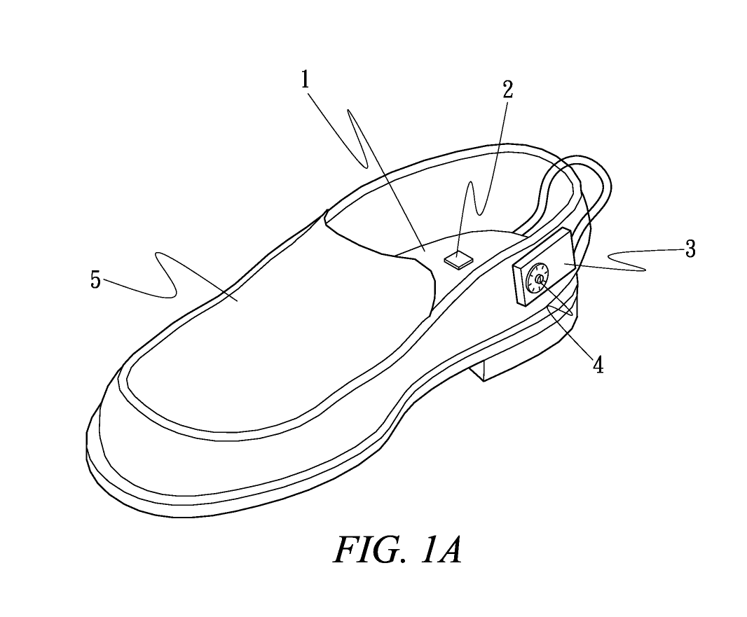

Referring to FIGS. 1, 1A, 2 and 3, the Fall-risk Evaluation and Balance Stability Enhancement System and method includes:

an insole 1 with stochastic resonance (SR) should be designed for the purpose of enhancing the stability of elderly adults with presumed fall risk. The criteria for generating appropriate stochastic resonance for different patients should be established.

a plurality of vibratory motor module 4 that is installed in insole 1;

a control module 5 which can further include a variable resister 10, installed on the shoes 5 that the insole 1 being inserted in.

For designing insoles 1, the effects of input noise on tactile sensation in humans need to be investigated. An important hypothesis is that the ability of an individual to detect a sub-threshold tactile stimulus can be significantly enhanced by the presence of a particular, nonzero level of noise. According to scientific correspondence [39], it is stated that “stochastic resonance, a phenomenon in which noise enhances the response of a nonlinear system to a weak signal has been demonstrated to be important functionally in various physiological systems”. And “these results suggest that a noise based technique could be used to improve tactile sensation in humans. Such a technique could be incorporated into the design of haptic interfaces for telerobotics and virtual environments”. From a clinical standpoint, a stochastic resonance-based technique could be applied to individuals with elevated coetaneous sensory thresholds, such as older adults and patients with peripheral neuropathies or strokes”. Furthermore, in the letter to nature [40], it is stated that “our experiment shows very clearly that weak signals can be enhanced by an optimal level of external noise in single sensory neurons. A recent psychophysical experiment and model involved human perception of ambiguous figures in the presence of external noise suggest that this is so”.

Relative setup for deriving the stochastic resonance, (SR) type and sub-sensory mechanical noise would be implemented. Sub-sensory mechanical noise will be applied to the soles of the feet via vibrating insoles. The vibratory shoes can be any kind of footwear that can be worn on patients, such as running shoes, slipper, or any kind of athletic, dress and casual or orthopedic shoes.

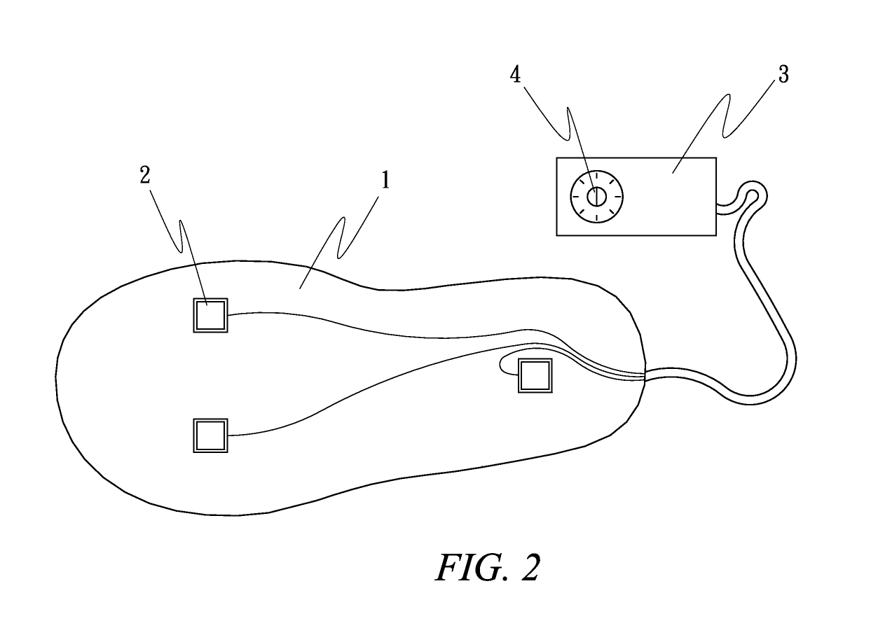

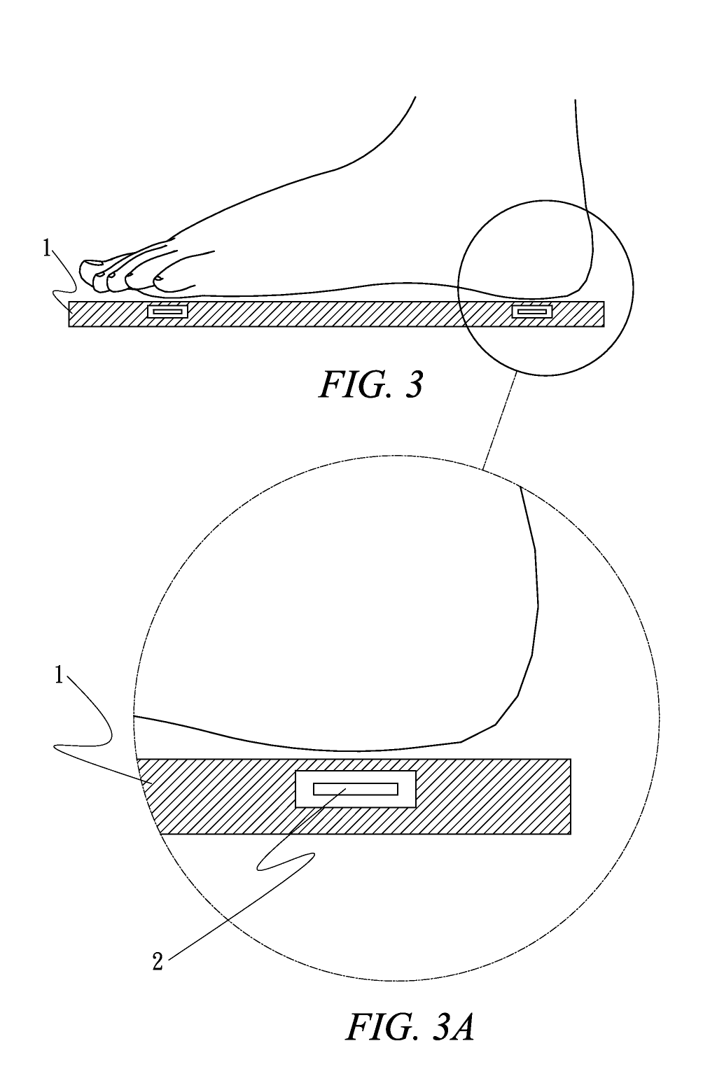

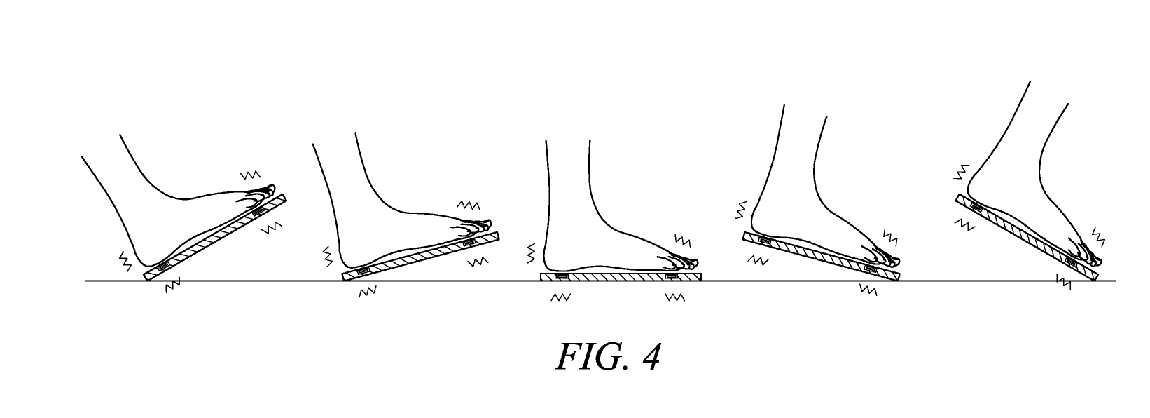

First, we put eccentric motor devices in the vibratory insoles 1. The eccentric motor 2 can be installed inside of insoles 1 as shown in FIG. 3 and FIG. 3A. The eccentric motors 2 are connected to the control module 3 to control the degree of vibration of the eccentric motors 2 as shown in FIG. 2, FIG. 3 and FIG. 3A. The subject can wear the footwear with the vibratory insole 1 at vibrating mode during the treatment process, as shows in FIG. 4. A variable resistor 4 can be installed on the control module 3 to adjust the degree of vibration intensity, so that subjects are able to feel comfortable to do the course of treatment.

(2) Assessment System

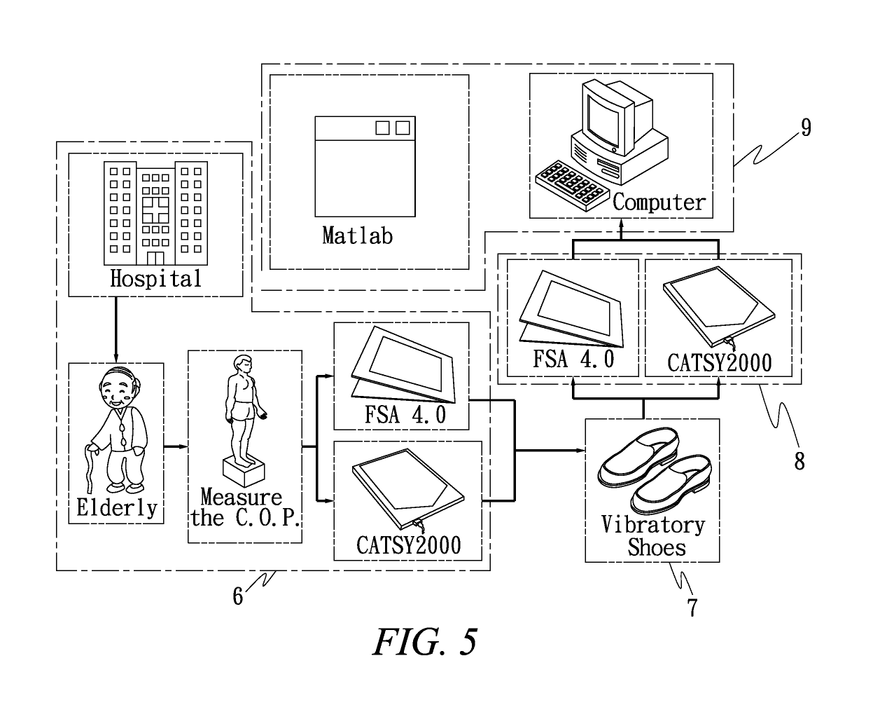

Before the experiment began, the subjects registered their self-basic information. As shown in FIG. 5, firstly, the subject puts on vibratory shoes on without turning on the vibration function. The subject sits on a chair. Following the instructor's instruction, the subject stands up and walks for 3 meters then turn back and walk straight back to the original chair and sit down. Therefore, the subject completes an up and go test. Next the subject would rest for a minute and stood on the non-invasive measurement physiological signals machine without wearing the vibratory shoes to collect the center of pressure (COP) data for 65 seconds (6) and then the subjects would be instructed to wear the vibratory shoes again. During the process, the subject will be fully protected by the personnel to prevent a fall due to poor balance of the subject.

After the COP test, subject is instructed to wear the vibratory shoes with the control module 5 turn on to produce vibration, which can be further controlled by a variable resister 10, to supply the physical stimulation and stimulate their feet of the nerve by adjusting the control module for maximum vibration that cannot be detected by subject. There are 6 different vibration degree controls (0, 10, 20, 30, 40, and 50) which are adjusted by changing the resistance value of variable resister 10. Zero means the largest vibration and 50 represents the lowest vibration. The variable resistance is adjusted to produce vibration that the subject is comfortable without any pinch or uncomfortable feeling. Subjects walked back and forth normally in a clear place for ten minutes (7). After the physical stimulation, the subjects take a rest for a minute and were allowed to stand on the non-invasive measurement physiological signals machine again with the vibratory shoes without turn on the vibration and complete an up and go test (8). After completing an up and go test, the subjects would rest for a minute and the center of pressure (COP) data is obtained by the non-invasive measurement physiological signals machine and sent the center of pressure (COP) data to the computer (9). We use Matlab computer software to write the multi-scale entropy (MSE) programs to perform analysis from the body center of pressure (COP) and compare with the differences of the body center of pressure (COP) between before and after the physical stimulation as shown in FIG. 5. We hoped the physical stimulation could improve the subjects' physical sense of balance and enhance the prediction of falls in the elderly, so that they could obtain more medical care and improve their balance and stability.

Since the center of pressure (COP) measurement apparatus obtains the body center of pressure (COP) data, the next step is via empirical mode decomposition (EMD) to detrend the data. After empirical mode decomposition (EMD), we use Multi-scale Entropy (MSE) to evaluate the center of pressure (COP). The details of EMD, entropy, and MSE are described as follows.

### (A) Empirical Mode Decomposition (EMD)

To calculate EMD, it is first realized that the EMD method is an iterative signal processing algorithm which decomposes the intrinsic components (IMFs) from signals by iterative sifting processes.

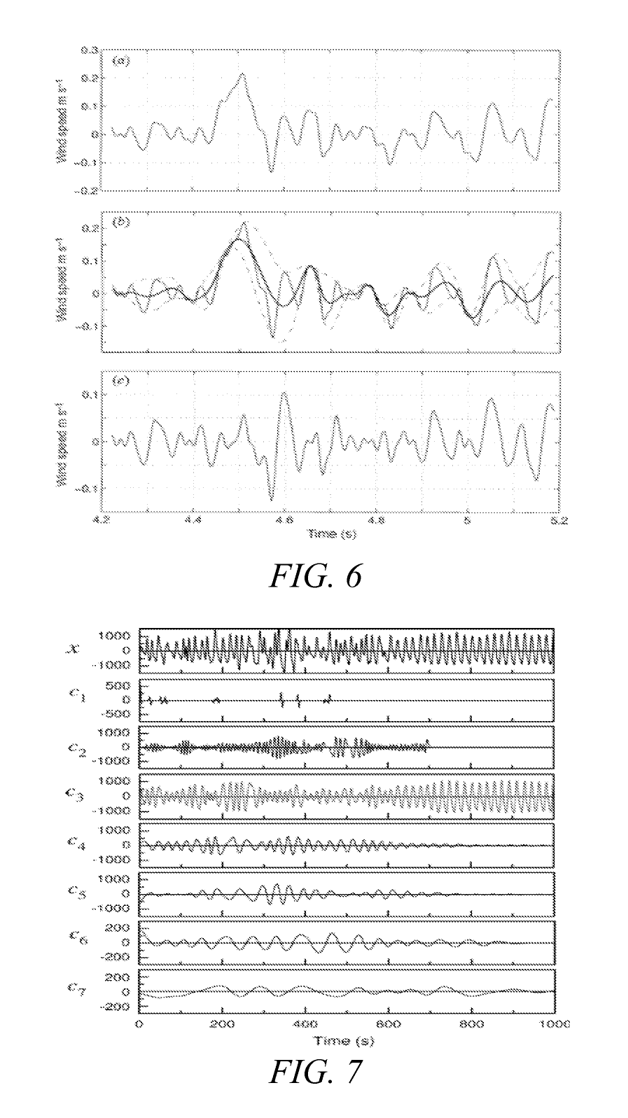

EMD was developed from the assumption that any time series data consist of different simple intrinsic modes of oscillation. The essence of the method is to identify the intrinsic oscillatory modes by their characteristic time scales in the data empirically, and then decompose the data accordingly. This is achieved by “sifting” data to generate IMFs show in FIG. 6. The IMFs obtained by EMD are a set of well-behaved intrinsic modes, and these functions satisfy the conditions that they are symmetric with respect to the local zero mean and have the same numbers of zero crossings and extremes.

The decomposition method can simply use the envelopes defined by the local maxima and minima separately. Once the extrema are identified, all the local maxima are connected by a cubic spline line as the upper envelope. Repeat the procedure for the local minima to produce the lower envelope. The upper and lower envelopes should cover all the data between them. Their mean is designated as m₁, and the difference between the data and m₁ is the first component h₁. Referring to FIG. 6, it illustration of the sifting processes: (a) the original data; (b) the data in thin solid line, with the upper and lower envelopes in dot-dashed lines and the mean in thick solid line; (c) the difference between the data and m₁. This is still not an IMF, for there are negative local maxima and positive minima suggesting riding wave.

X(t)−m₁=h₁. (1)

The resulting component h₁ is an IMF if it satisfies the following conditions: (I) h₁ is free of riding waves; (II) it displays symmetry of the upper and lower envelopes with respect to zero; (III) the numbers of zero crossing and extremes are the same, or only differ by 1. Besides these, an additional condition based on the intermittence (i.e., varying numbers of data points per cycle; see the explanation below) can be imposed here to sift out waveforms with a certain range of intermittence for physical consideration. If h₁ is not an IMF, the sifting process has to be repeated as many times as is required to reduce the extracted signal to an IMF.

In the subsequent sifting process steps, h₁ is treated as the data to repeat the steps mentioned above.

h₁−m₁₁=h₁₁. (2)

Again, if the function h₁₁ does not yet satisfy criteria, the sifting process continues up to k times until some acceptable tolerance are reached:

h₁₍ₖ₋₁₎−m₁ₖ=h₁ₖ (3)

If the resulting time series is the first IMF, it is designated as c₁=h₁ₖ. The first IMF is then subtracted from the original data, and the difference r₁ given by

X(t)−c₁=r₁ (4)

Since the residue, r₁, still contains information of longer period components, it is treated as the new data and subjected to the same sifting process as described above. This procedure can be repeated on all the subsequent rⱼs, and the result is

r₁−c₂=r₂, . . . , rₙ₋₁−cₙ=rₙ. (5)

The sifting process can be stopped by any of the following predetermined criteria: either when the component, cₙ, or the residue, rₙ, becomes so small that it is less than the predetermined value of substantial consequence, or when the residue, rₙ, becomes a monotonic function from which no more IMF can be extracted. Even for data with zero mean, the final residue can still be different from zero; for data with a trend, then the final residue should be that trend. FIG. 7 shows typical results from EMD and the resulting empirical mode decomposition components from the typical time series data. By summing up equations (4) and (5), we finally obtain the equation (6).

$$X ( t ) = {\sum }_{n = 1}^{N} {C}_{n} + {r}_{n} \qquad ( 6 )$$

Also, in biological science or engineering, entropy estimation of biological signal has often been performed as an important analytical tool. Entropy is an important measure utilized not only in information theory, but also in a wide variety of scientific fields including biological signal processing [22], [23], and [24].

### (B) Entropy

Calculation of the entropy of a set of consecutive order statistics is relatively more complicated than that of the entropy of the individual order statistic. The entropy of a set of data is a measure of the amount of information contained in it. Entropy calculations for fully specified data have been used to get a theoretical bound on.

Classical entropy and physiologic complexity concepts do not have a straightforward correspondence. Entropy is related to the degree of “randomness” of a time series and it maximize for completely uncorrelated random signals. Complexity is related to the underlying structure of a time series and its information content. An increase of the entropy assigned to a time series usually, but not always, corresponds to an increase of underlying system complexity.

A diseased system, when associated with the emergence of more regular behavior, show reduced entropy values in comparison to the dynamics of free-running healthy systems [25]. Traditional algorithms will yield an increase in entropy values for such noisy. Pathologic signals in comparison to healthy dynamics are showing correlated (1/f type, where f represents signal frequency) properties, even though the latter represent more physiologically complex and adaptive states. This inconsistency may be related to the fact that widely used entropy measures are based on signal-scale analysis and do not take into account the complex temporal fluctuations inherent in a healthy physiologic control system.

The entropy H(X) of a single discrete random variable X is a measure of its average uncertainty. Entropy is calculated by the equation:

$$H ( X ) = - {\sum }_{{x}_{i} ∈ Θ}^{ } p ( {x}_{i} ) log p ( {x}_{i} ) \qquad ( 7 )$$

where X represents a random variable with set of values Θ and probability mass function p(xᵢ).

For a time series representing the output of a stochastic process, that is, an indexed sequence of n random variables, {Xᵢ}={Xᵢ, . . . , Xₙ} with the set of values Θ₁, . . . Θₙ, respectively, the joint entropy is defined as

$${H}_{n} = - {\sum }_{{x}_{i} ∈ {Θ}_{1}}^{ } … {\sum }_{{x}_{i} ∈ {Θ}_{n}}^{ } p ( {x}_{1} , … , {x}_{n} ) log p ( {x}_{1} , … , {x}_{n} ) \qquad ( 8 )$$

where p(x₁, . . . , xₙ) is the joint probability for the n variables X₁, . . . , Xₙ.

Numerically, only entropies of finite order n can be computed. As soon as n becomes large with respect to the length of a given time series, the entropy underestimated and decay towards zero. Therefore, the Kolmogorov-Sinai (KS) entropy for “real-world” time series of finite length cannot usually be estimated with reasonable precision.

For the analysis of such typically short, noisy time series, Pincus [26] introduced the approximate entropy (ApEn) family of parameters, which have been widely used in physiology and medicine. Recently, a modified algorithm, sample entropy (SampEn) [27], has been proposed, which has the advantage of being less dependent on the time series length. Such algorithms, however, assign a higher value of entropy to certain pathologic time series that are presumed to represent fewer complex dynamics than to time series derived from healthy function [25]. One possible reason for obtaining these results may be the fact that these measures are based on a single scale. Both the KS entropy and the related ApEn parameters depend on a function's one step different (e.g., Hₙ₊₁-Hₙ) and reflect the uncertainty of the next new point, given the history of the series.

### (C) Multi-Scale Entropy, MSE

Entropy-based algorithms for measuring the complexity of physiologic time series have been widely used. They have proven to be useful in discriminating between healthy and disease slates, although some results may generate misleading conclusions. For example, the entropy that these algorithms assign to time series derived from the ventricular response in atrial fibrillation (AF) is higher than that assigned to sinus rhythm time series derived from healthy subjects. However, healthy systems generate much more complex outputs than diseased ones. Traditional algorithms are single-scale based and, therefore, fail to account for the multiple time scales inherent in physiologic systems.

Zhang [28, 29] propose a general approach to take into account the multiple time scales in a physical system. His measure, based on a weighted sum of scale dependent on entropies, does, in fact, yield higher values for correlated noise in comparison to uncorrelated ones. However, since it is based on Shannon's definition of entropy, Zhang's method requires a large amount of almost noise-free data in order to map a signal to a discrete symbolic sequence with sufficient statistical accuracy. Therefore, it presents obvious limitations when applied to typical physiologic signals that vary continuously and have been finite.

A one-dimensional discrete time series is {Xᵢ, . . . , Xₙ}. It construct consecutive coarse-grained time series, {y^{(τ)}}, determined by the scale factor, τ, according to the equation :

yⱼ^{(τ)}=1/τΣ_{i=(j-1)τ+1}^{jτ}xᵢ 1≦j≦N/τ (9)

For scale one, the time series {y⁽¹⁾} is simply the original time series. The length of each coarse-grained time series is equal to the length of the original time series divided by the scale factor, τ. Calculate an entropy measure (SampEn) for each coarse-grained time series plotted as a function of the scale factor τ [30]. It called this procedure of multi-scale entropy (MSE) analysis [31].

### Data Analysis

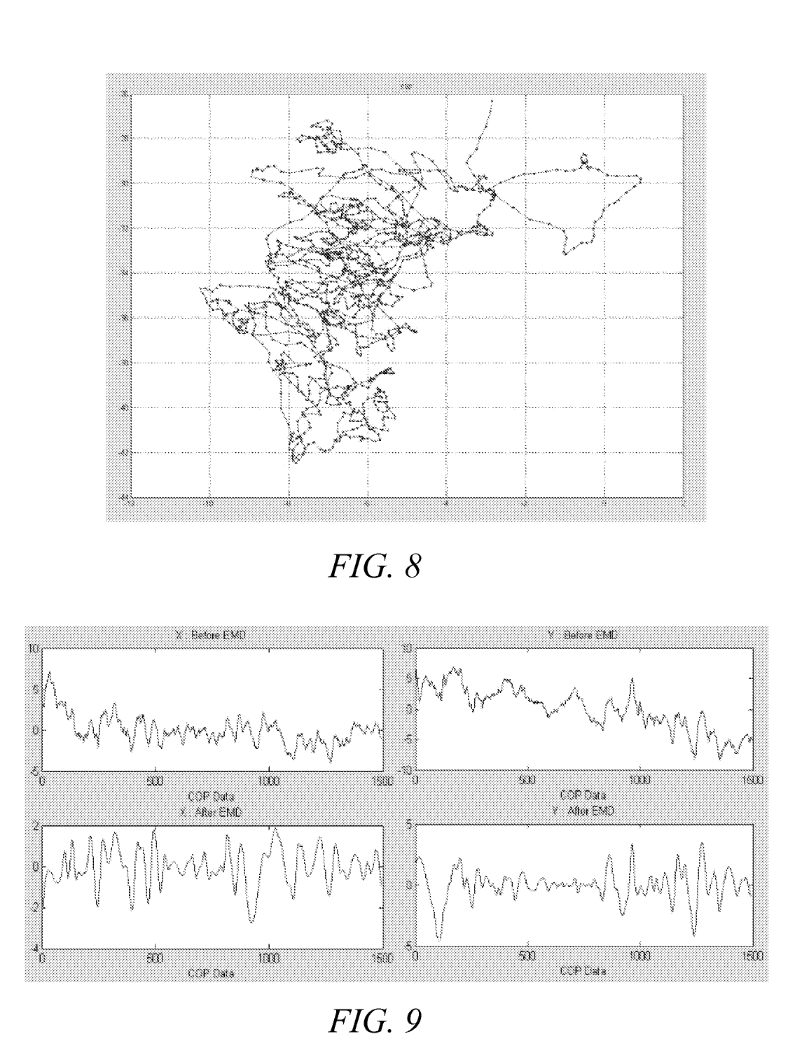

In this study, the subject's center of pressure (COP) signal data were recorded from the CATSYS2000 as shown in FIG. 8, First of all, we used the empirical mode decomposition (EMD) to detrend and decompose the center of pressure (COP) signal data, as shown in FIG. 9.

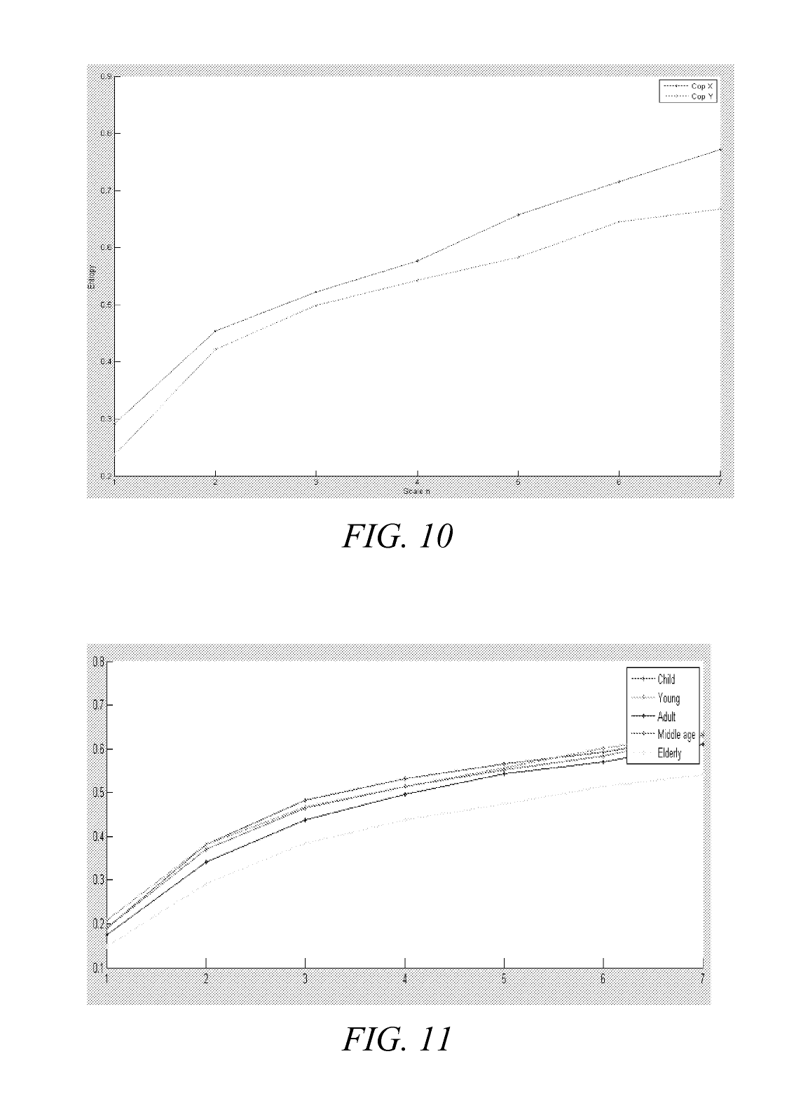

Second, we use the multi-scale entropy (MSE) to analysis the signals after EMD. FIG. 10 is a multi-scale entropy (MSE) value graph and the scale factor is 7.

### Results

### (1) Assessment System

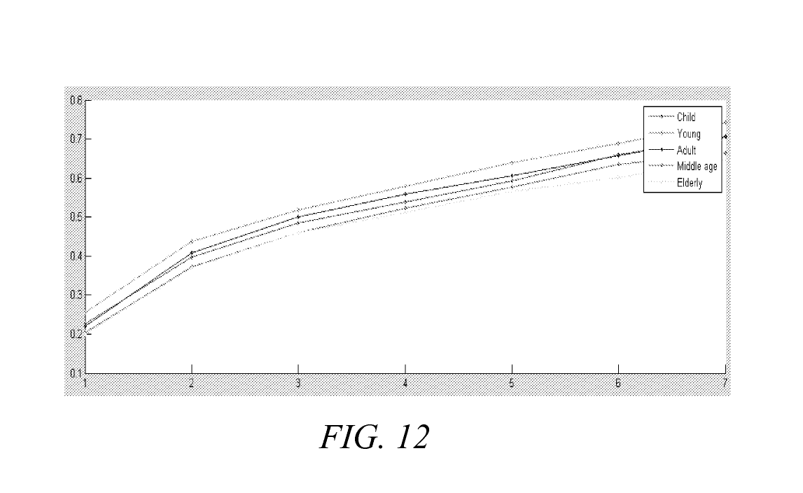

After finished the multi-scale entropy (MSE) and record all the subjects' results as shown in FIG. 11 and FIG. 12. In FIG. 11, for each scale factor, the MSE value in the elderly group is the lowest. This shows the complexity of the elderly is lower, and that they have a poor sense of balance in the horizontal direction. However, the MSE value in the children group is the highest. It also shows the complexity of the children group is highest, and that they have a good sense of balance in the horizontal. FIG. 11 is the MSE analysis of the center of pressure from five groups with different age, Analysis of the data for the horizontal (X-axis). Subjects divided into children (under 20-year-old age), youth (20˜40 years), adults (40˜50 years), middle-aged (50˜60 years old) and elderly (over sixty-year-old above). In FIG. 12, for each scale factor, the MSE value in the elderly group is the lowest. This shows the complexity of the elderly group is lower, and that they have a poor sense of balance in the vertical. The MSE value in young group is highest and shows the complexity of the young group is higher, and that they have a good sense of balance in the vertical.

Taking the average area of the MSE values in X-axis and Y-axis and calculating the standard deviation to each group in the following table, it can show the Young group has the higher values of the average area of the MSE values, whether in X-axis or Y-axis. In addition, the elderly group has the lowest values of the average area of the MSE value.

| | Elderly | Middle age | Adult | Young | Child |
|---|---|---|---|---|---|
| Area X | 2.444 | 2.896 | 2.786 | 2.941 | 2.963 |
| Standard Deviation | 0.113 | 0.118 | 0.123 | 0.118 | 0.119 |
| Area Y | 2.932 | 3.000 | 3.197 | 3.362 | 3.141 |
| Standard Deviation | 0.124 | 0.133 | 0.133 | 0.134 | 0.134 |

The analysis results of the using MSE illustrated that the elderly group gets the lowest values, whereas the young group gets the highest values. This fully explained the results of the analysis complied with the general concept that usually consider the sense of balance of the young group is better than the elderly group because of the increase of age and declines the sense of a balance phenomenon. We observe the MSE also has this trend. Therefore, we conclude that the MSE analysis method could efficiently assess the body balance of human via the center of pressure (COP) signals.

### (2) Treatment System

The experiment procedure is measuring the body center of pressure (COP) signals at the first by putting on the vibratory shoes and using the physical stimulate to therapy the sense of body balance and then measuring the center of pressure (COP) signals again. Then we will compare the results with these two centers of pressure (COP) data.

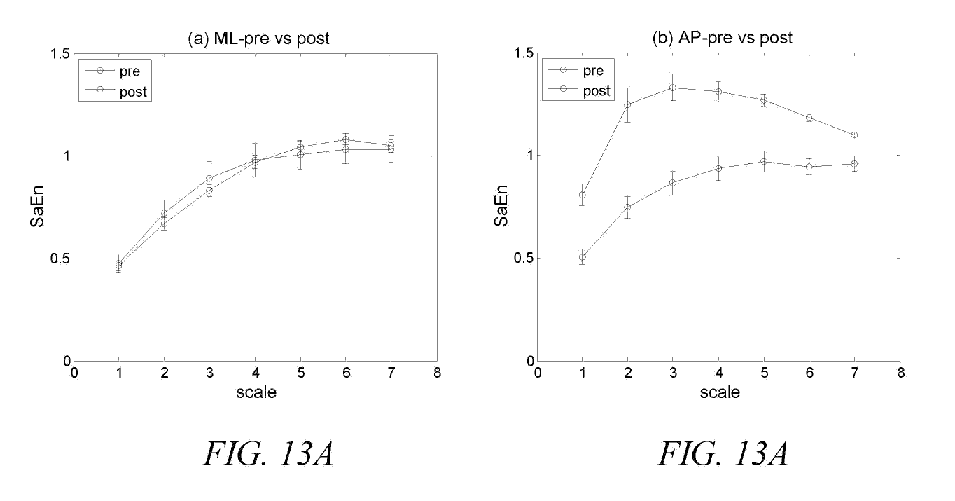

The experiment collected the MSE data from 8 elderly subjects and analyzed the center of pressure (COP) signal. We made an index in MSE area and observe the change in MSE area during before and after putting on the vibratory shoes. In the fall of the main factors, most fall behaviors are land on forward or backward (vertical axis) and the left or right side of the falls are relatively less. In the fall of our analysis, we focus on the vertical axis (Y-axis) and horizontal axis (X-axis) as a supplement. One of the subjects measured to the center of pressure (COP) signal for MSE analysis of the results can be seen at X-axis and Y-axis as shown in FIG. 13A, 13B. The previous sections have mentioned that calculate of the area of MSE is able to know the degree of complexity.

The 8 elderly subjects measured to the center of pressure signal for MSE analysis of the results can be seen at X-axis, mediolateral (ML) and Y-axis anteroposterior (AP) as shown in following table with comfortable resistance sitting.

| sub- | Resistance | ML direction | | | AP direction | | |
|---|---|---|---|---|---|---|---|
| ject | (ohms) | before | after | result | before | after | result |
| 1 | 15 | 6.1313 | 6.7029 | better | 5.6556 | 8.629 | better |
| 2 | 35 | 6.6144 | 6.5622 | No | 6.1877 | 8.2157 | better |
| | | | | Change | | | |
| 3 | 0 | 5.9893 | 7.2642 | better | 7.0117 | 9.492 | better |
| 4 | 10 | 5.4228 | 5.693 | No | 5.5208 | 8.2426 | better |
| | | | | change | | | |
| 5 | 0 | 6.0539 | 6.1015 | No | 4.4705 | 7.5678 | better |
| | | | | change | | | |
| 6 | 10 | 6.4596 | 5.2485 | worsen | 6.4446 | 8.3568 | better |
| 7 | 0 | 6.3602 | 4.0661 | worsen | 6.2318 | 7.2875 | better |
| 8 | 10 | 5.8219 | 7.4329 | better | 5.8861 | 8.1521 | better |

The first area is the MSE area before stimulation and the second area is the MSE area after stimulation. By observing of changes in the MSE area is to discuss the changes in complexity as shown in following table.

| Complexity index | ML direction | AP direction |
|---|---|---|
| Before the test | 6.107 ± 0.380 | 5.926 ± 0.753 |
| After the test | 6.134 ± 1.117 | 8.234 ± 0.665 |
| Paired-t test | 0.953 | <0.001 |

From above table, if the second area value is larger than the first area value, it means the second degree of complexity is higher than the first degree of complexity and the body sense of balance becomes better temporarily. After testing 8 elderly subjects, 3 elderly subjects of degree of complexity become better, 2 elderly subjects become worse and 3 elderly subject had no significant difference in X-axis (horizontal, mediolateral (ML)). In Y-axis (vertical, anteroposterior (AP)), the degree of complexity of 8 elderly subjects becomes better, 0 elderly subjects become worse and 0 elderly subject had no significant difference.

The result of the experiment is shown in following table.

| | Subjects | Better | Worse | No Significant difference |
|---|---|---|---|---|
| In X-axis | 8 | 3 | 2 | 3 |
| In Y-axis | 8 | 8 | 0 | 0 |

Hence, the degree of vibration intensity in vibratory shoes will affect the value of complexity.

The assessing system of the center of pressure (COP) can figure out the subject's sense of the balance condition. By collecting more subjects' center of pressure (COP) signals, we could obtain the average value of each group to form an index for this assessment system. Furthermore, by comparing the subject's the center of pressure (COP) signal with every group's index, we could know if the subjects have any problems with body balance.

We installed the variable resistors in vibratory shoes, and we could adjust the intensity of stimulation to precede the experiment within the acceptable limit for each subject, so that subjects will feel better during the experimental process. And the subjects have safe and carefree conditions to do the treatment center of the pressure (COP) experiments.

Stochastic resonance, a phenomenon in which noise enhances the response of a nonlinear system to a weak signal [32-33], has been demonstrated to be important functionally in various physiological systems [34-38]. Adding appropriate noise is useful to the physiological system. We defined the vibration as a kind of noise and controlled the degree of vibration intensity via a variable resistor and calculate the electric power of vibratory shoes as a noise (N) according to the different degree of noise divided into five parts as shown in the following table.

| N = V/R² | X | Y | |
|---|---|---|---|
| N1 = 0 | 4.532988 | 4.637892 | No stimulate |
| N2 = 0.225 | 4.979452 | 5.482755 | R = 40Ω |
| N3 = 0.45 | 4.963912 | 5.44987 | R = 20Ω |
| N4 = 4.5 | 4.758888 | 5.70111 | R = 2Ω |
| N5 | 2.982862 | 4.062021 | No insole |

We obtained the MSE area values in five noises from one subject as shown in

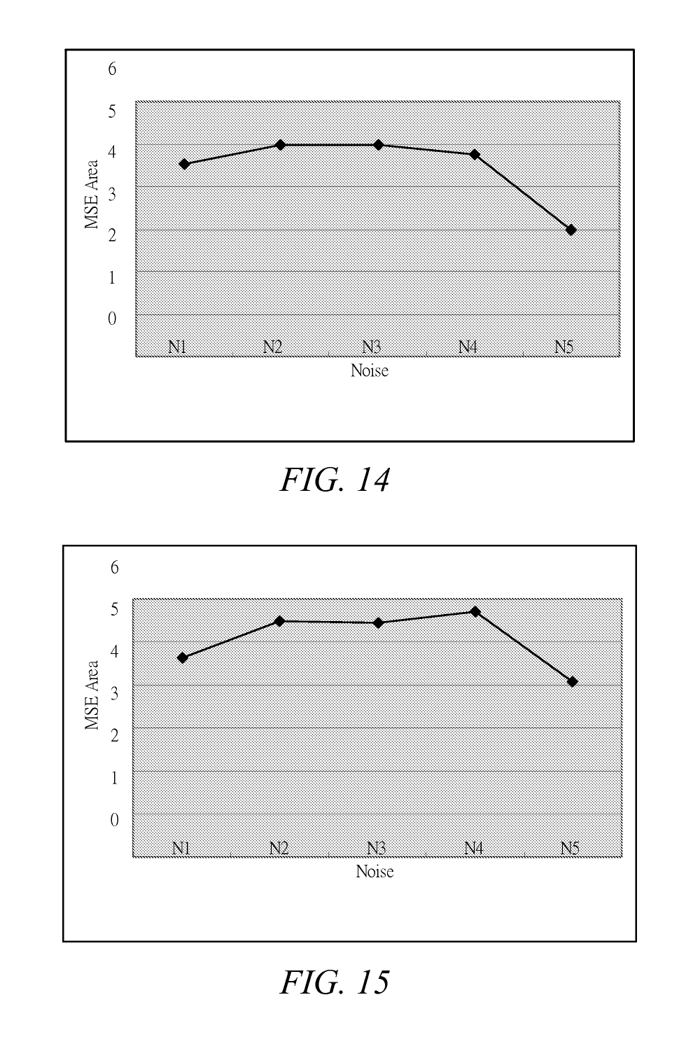

FIG. 14, and FIG. 15.

In X-axis (horizontal), the MSE area values in N2 (40Ω)-N3 (20Ω) and N4 (2Ω) had no significant difference, but they are also better than N1 and N5. In Y-axis (vertical), the MSE area value is highest in N4 (2Ω), N2 (40Ω) and N3 (20Ωand no significant difference but they are also better than N1 and N5. N1 is the noise before stimulate and N5 is the noise had no insole. If the subjects wore the vibratory shoes with no insole during the walk, they felt very uncomfortable and even did not want to wear the vibratory shoes anymore.

This result indicates that the appropriate stimulation of vibratory is helpful to the body balance system, but the excessive stimulation of vibratory causes the decline in the body balance system. This inference is consistent with prior results of the proposed. When subjects wear the vibratory shoes and proceed with the experiment, it the degree of vibration intensity should be adjusted by a variable resistor first and subjects would not feel uncomfortable, so that the vibratory shoes can be the effect of the treatment of physical sense of the balance system.

While there have been shown preferred embodiment of a dynamic system in accordance with the invention, it is to be understood that many changes may be made therein without departing from the spirit of the invention.

### REFERENCES

[1] B. S. Roudsari, B. E. Ebel, P. S. Corso, N. M. Molinari, T. D. Koepsell, The acute medical care costs of fall-related injuries among the U.S older adults, J. Care Injured (2005) 36, 1316-1322.
[2] E. Zaloshnja, T. R. Miller, B .A. Lawrence, E Romano, The costs of unintentional home injuries. J. of Preventive Medicine, 2005, 28(1).
[3] Survey of population in Taiwan, report of department of statistics, ministry of the interior Taiwan, http://www.moi.gov.tw/stat/index.asp.
[4] Statistical report of top ten causes of death in Taiwan, report of department of health, Exclusive Yuan, R.O.C. (Taiwan), http://www.doh.gov.tw/statistic/.
[5] C. A. Laughton, M. Slavin, K. Katdare, L. Nolan, J. F. Bean, D.C. Kerrigan, E. Phillips, L. A. Lipsitz, K. J. Collins, Aging, muscle activity, and balance control: physiological changes associated with balance impairment, Gait and Posture 18 (2003), 101-108.
[6] D. G. Miceli, Falls associated with Dementia: How can you tell? Geriatric Nursing, Vol. 26, Number 2. 2005; 26:106-10.
[7] A. Gefen, “Simulations of Foot Stability During Gait Characteristic of Ankle Dorsiflexor Weakness in the Elderly”, S 1534-4320(01)11421-X.
[8] D. A. Schieb, “Walkway Surface Heights and Ground Reaction Forces”, 0-7803-:2083-2/95 1995 IEEE
[9] R. A. Cooper, D. P. VanSickle, R. N. Robertson, M. L. Boninger and G. J. Ensminger, “A Method for Analyzing Center of Pressure During Manual Wheelchair Propulsion”, IEEE, vol. 3, no. 4, December 1995
[10] P. R. cavanagh, “L technique for averaging center of pressure paths from a force platform”, J. Biomechanics, vol. 11, pp. 487491, 1978.
[11] B. J. Benda, P. O. Riley and D. E. Krebs, “Biomechanical Relationship Between Center of Gravity and Center of Pressure During Standing”, IEEE, vol. 2, no. 1, March 1994
[12] O. Sezer and M. Ferdjallah, “Adaptive Autoregressive Model for the Analysis of Center of Pressure in Healthy Subjects during Quiet Standing”, 0-7803-9197-7/05/ 2005 IEEE.
[13] H. Snoussi, H. Amoud, M. Doussot, D. Hewson and J. Ducĥene, “Reconstructed Phase Spaces of Intrinsic Mode Functions. Application to Postural Stability Analysis”, 1-4244-0033-3/06/ 2006 IEEE.
[14] S. W. Yip and T. E. Prieto, “A system for force distribution measurement beneath the feet”, 0-7803-3 13 1-1/96 1996 IEEE
[15] S. Park, F. B. Horak, A. D. Kuo, “Postural feedback responses scale with biomechanical constraints in human standing”, Exp Brain Res 2004, 154:417-427.
[16] E. P. Zehr, J. Duysens, “Regulation of arm and leg movement during human locomotion”, Neuroscientist 2004, 10:347-361.
[17] B. E. Maki, W. E. McIlroy, “Postural control in the older adult”, Clin Geriatr Med 1996, 12:635-658.
[18] H. Tropp, Commentary: Functional ankle instability revisited. J Athl Train 2002, 37:512-515.
[19] A. Kavounoudias, R Roll, J. P. Roll: Specific whole-body shifts induced by frequency-modulated vibrations of human plantar soles. Neurosci Lett 1999, 266:181-184.
[20] A. Kavounoudias, R. Roll, J. P. Roll, “Foot sole and ankle muscle inputs contribute jointly to human erect posture regulation”. J Physiol 2001, 532:869-878.
[21] P. Novak and V. Novak, “Effect of step-synchronized vibration stimulation of soles on gait in Parkinson's disease: a pilot study”, Journal of NeuroEngineering and Rehabilitation 2006, 3:9
[22] R. R. de R. van Steveninck, G. D. Lewen, S. P. Strong, R. Koberle, and W. Bialek, “Reproducibility and variability in neural spike trans,” Science, vol. 275, no. 21, pp. 1805-1808,1997.
[23] S. P. Strong, R. Koberle, R. R. de R. van Steveninck, and W. Bialek,“Entropy and information in neural spike trains,” Phys. Rev. Lett., vol. 80, no. 1, pp. 197-200, 1998.
[24] S. R. Schultz and S Panzeri, “Temporal Correlations and Neural Spike Train Entropy,” Phys. Rev. Lett., vol. 86, no. 25, pp. 5823-5826, June 2001.
[25] A. L. Goldberger, C.-K Peng, and L. A. Lipsitz, What is physiologic complexity and how does it change with aging and disease?, Neurobiol. Aging, January 2002 (Vol. 23, Issue 1, Pages 23-26).
[26] S. M. Pincus, Approximate entropy as a measure of system complexity. Proc Natl Acad Sci USA. 1991 Mar. 15; 88(6): 2297-2301.
[27] J. S. Richman and J. R. Moorman, Physiological time-series analysis using approximate entropy and sample entropy, Am. J. Physiol. Vol. 278, Issue 6, H2039-H2049, June 2000.
[28] Y.-C. Zhang. Complexity and 1/f noise. A phase space approach. Journal de Physique I, Volume 1, Issue 7, July 1991, pp. 971-977
[29] H. C. Fogedby. On the phase space approach to complexity. Journal of Statistical Physics, Volume 69, Issue 1-2, pp. 411-425
[30] A. L. Goldberger and C.-K. Peng, Multi-scale Entropy Analysis of Complex Physiologic Time Series, Physical Review Letters, DOI: 10.1103/PhysRevLett.89.068102
[31] J. L. Starck, F. Murtagh, and A. Bijaoui, Image Processing and Data Analysis Cambridge University Press, Cambridge, (1998).
[32] K. Wilesenfeld and F. Moss, “Stochastic resonance and the benefits of noise: from ice ages to crayfish and SQUIDs”, Nature 373, 33-36 (1995)
[33] J. J. Collins, C. C. Chow and T. T. Imhoff, “Stochastic Resonance Without Tuning”, Nature 376, 236-238 (1995)
[34] J. K. Douglass, L. Wilkens, E. Pantazelou and F. Moss, “Noise enhancement of information transfer in crayfish mechanoreceptors by stochastic resonance”, Nature 365, 337-340 (1993)
[35] J. E. Levin and J. P. Miller, “Broadband Neural Encoding in the Cricket Cercal Sensory System Enhanced by Stochastic Resonance.”, Nature 380, 165-168 (1996)
[36] J. J. Collins, T. T. Imhoff and P. J. Grigg, “Noise-enhanced information transmission in rat SA1 cutaneous mechanoreceptors via aperiodic stochastic resonance”, Neurophysiol. 76, 642-645 (1996)
[37] P. J. Cordo, “Noise in human muscle spindles”,Nature 383, 769-770 (1996)
[38] J. T. Inglis, S. Verschueren, J. J. Collins, D. M. Merfeld, S. Rosenblum, S. Buckley and F. Moss, “Noise-enhanced tactile sensation”, Nature Vol. 383 (1996)
[39]Paul Cordo, Nature 383, 770, 31 Oct. 1996
[40]John K. Douglass, Lon Wilkens, Elenl Pantazelou & Frank Moss, Nature, 365 337-339, 23 Sep. 1993

What is claimed is:

1. A Fall-risk Evaluation and Balance Stability Enhancement System including:
an insole with different sizes to fit in different type of footwear;
a plurality of vibratory motor modules that is installed in insole for generating appropriate stochastic resonance for different patients;
a control module which installed on the footwear that the insole being inserted in and connected to the vibratory motor modules;

2. A system as set froth in claim 1, in which control module further includes variable resistors for adjusting vibration.

3. A system as set froth in claim 1, in which vibratory motor modules are eccentric motors.

4. A system as set froth in claim 1, in which connection between vibratory motor modules and control module is setup at back side of the insole.

5. A system as set froth in claim 1, in which connection between vibratory motor modules and control module is setup in insole.

6. A fall-risk evaluation and balance stability enhancement method including following steps:
(a) the subjects stood wearing vibratory shoes on the non-invasive measurement physiological signals machine to collect the center of pressure (COP) data;
(b) vibratory shoes to supply the physical stimulation and stimulate their feet of the nerve and subjects walked back and forth normally in a clear place;
(c) after the physical stimulation, the subjects were allowed to stand on the non-invasive measurement physiological signals machine again;
(d) obtained the center of pressure (COP) data by the non-invasive measurement physiological signals machine and returned the center of pressure (COP) data to the computer and compare with the differences of body center of pressure (COP) between before and after the physical stimulation.

7. A method as set froth in claim 6, in which non-invasive measurement physiological signals machine uses empirical mode decomposition (EMD) to detrend the data then use Multi-scale Entropy (MSE) to evaluate the center of pressure (COP).
